(19) United States
(12) Patent Application Publication
(10) Pub. No.: US 2024/0142382 A1
(43) Pub. Date: May 2, 2024

(54) METHODS AND COMPOSITIONS FOR CALIBRATED LABEL-FREE SURFACE-ENHANCED RAMAN SPECTROSCOPY

(71) Applicant: Virginia Tech Intellectual Properties, Inc., Blacksburg, VA (US)

(72) Inventors: Wei ZHOU, Blacksburg, VA (US); Wonil NAM, Blacksburg, VA (US)

(21) Appl. No.: 18/280,452

(22) PCT Filed: Mar. 4, 2022
(86) PCT No.: PCT/US2022/018832

§ 371 Date: Sep. 5, 2023

Related U.S. Application Data
(60) Provisional application No. 63/157,589, filed on Mar. 5, 2021.

(51) Int. Cl. G01N 21/65; G01N 33/483

(52) U.S. Cl. CPC G01N 21/658; G01N 33/4833; G01N 2201/06113

(57) ABSTRACT

In one aspect, the disclosure relates to methods of label-free surface-enhanced Raman spectroscopy (SERS) comprising plasmonically enhanced electronic Raman scattering (ERS) signals from metal nanostructures as an internal calibration standard to improve multivariate analysis of living biological systems, and the uses of the methods therein. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/157,589, filed on Mar. 5, 2021, which is incorporated herein by reference in its entirety.

### STATEMENT REGARDING FEDERALLY SPONSORED RESEARCH

This disclosure was made with U.S. Government support under grant FA9550-18-1-0328 awarded by the Air Force Office of Scientific Research. The U.S. government has certain rights in the disclosure.

### BACKGROUND

Rapid molecular-level characterization of biological samples is highly desirable for identifying biomarkers related to specific diseases and pathogens, characterizing cellular responses in drug testing, and screening up- or down-regulated pathways, but remains challenging in biology and medicine. Currently, mass spectrometry and nuclear magnetic resonance spectroscopy are the two gold standard methods to provide quantitative structural information for complex biological samples. Nevertheless, these measurements are destructive and time-consuming, suitable for the end-point analysis but not for real-time monitoring of dynamic changes in living biological systems. As a promising alternative, surface-enhanced Raman spectroscopy (SERS) has emerged as a label-free, non-destructive, and rapid detection technique to provide vibrational molecular fingerprint information of biological samples without water vibrational interference. Notably, by surface plasmon enhancement of both excitation and inelastic scattering processes for molecules at plasmonic hotspots, the sensitivity of SERS can reach a single-molecule detection limit. Molecular specific and label-free SERS approaches can allow the detection of specific biomolecules (e.g., metabolites, amino acids, proteins, and nucleic acids) in complex matrices (e.g., food, blood plasma, serum specimens, and body fluids) as well as the investigation of dynamic biological processes in living biological systems (e.g., cell cultures, tissues, and animal models).

Despite significant advances, label-free SERS analysis of living cells still faces challenges in reliability and reproducibility mainly because SERS signals are incredibly susceptible to local optical field variations at plasmonic hotspots. A promising method for calibrating SERS signals is to generate internal standards from reference tag molecules incorporated at hotspots, and the calibrated SERS signals can more accurately reflect the actual biomolecule concentration. However, the tag molecule-based internal standards suffer significant limitations due to (i) poor photostability under laser excitation, (ii) spectral interference with Raman signatures of biomolecule matrices, and (iii) adsorption competition with biomolecules at hotspots, especially detrimental to chronic living cell measurements and analysis. To overcome the aforementioned limitations of tag molecule-based SERS calibration, the present disclosure relates to methods of using plasmon-enhanced electronic Raman scattering (ERS) signals from metal nanostructures as an internal calibration standard for label-free SERS.

### SUMMARY

In accordance with the purpose(s) of the disclosure, as embodied and broadly described herein, the disclosure, in one aspect, relates to a method for label-free surface-enhanced Raman spectroscopy (SERS) of complex biological samples as a rapid non-destructive molecular fingerprint characterization technique. In various further aspects, the disclosed methods use plasmonically enhanced electronic Raman scattering (ERS) signals from metal nanostructures as a SERS calibration internal standard (“ERS-Calibrated SERS”). In various further aspects, the disclosed methods can be used for subtyping degrees of malignancy cancer cells using the same. In various further aspects the disclosed methods can be used to assess cellular drug responses at varying dosages.

Disclosed are methods of label-free SERS of cells, the method comprising: providing a sample system, wherein the sample system comprises a nanolaminated SERS substrate, and wherein a plurality of cells are adherent to at least one surface of the nanolaminated SERS substrate; carrying out plasmonically enhanced ERS calibration; obtaining a dataset comprising second SERS measurements over a dataset mapping area; and subjecting the dataset to multivariate analysis.

Also disclosed are methods of characterizing biological specimens, which may involve identifying a cell type or state corresponding to a disease or health condition of a subject. Also disclosed are methods of subtyping cancer cells by degrees of malignancy using ERS-calibrated SERS.

Also disclosed are methods of assessing exogenous impact on cells using ERS-calibrated SERS.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described aspects are usable in all aspects of the disclosure taught herein. Furthermore, the individual features of the dependent claims, as well as all optional and preferred features and modifications of the described aspects are combinable and interchangeable with one another.

### BRIEF DESCRIPTION OF THE FIGS.

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

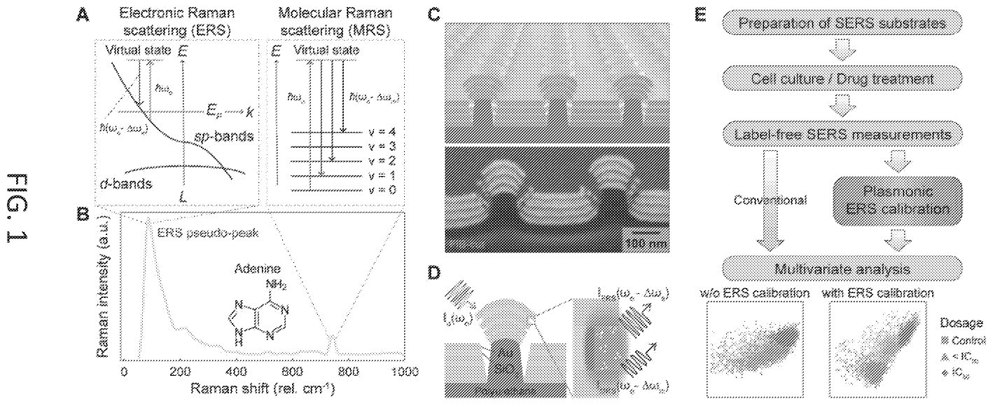

FIG. 1 shows representative data for ERS signals in SERS measurements and ERS calibration for label-free living cell SERS biostatistical analysis. (A) Energy-diagram illustration of the ERS process (left) and the MRS process (right). (B) A representative SERS spectrum using adenine molecules, showing the ERS pseudo-peak and MRS signals. (C) Schematic illustration of nanolaminated SERS substrates (top) and corresponding cross-sectional scanning electron microscope (SEM) image achieved by FIB milling. (D) Schematic illustration of the ERS and MRS processes at plasmonic hotspots in a unit cell of nanolaminated SERS substrates. (E) A flow diagram of the major steps for ERS-calibration-enabled improved multivariate analysis of living cell SERS.

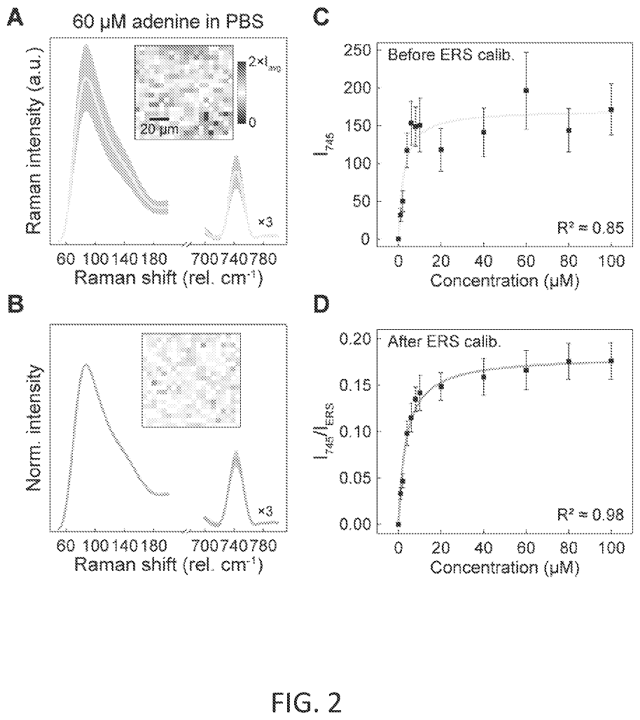

FIG. 2 shows representative data for ERS calibration for quantitative SERS analysis of solution-based adenine molecules. (A-B) Averaged SERS spectrum of 60 μM adenine solution with SD (gray shaded regions) (A) before and (B) after ERS calibration. MRS region between 700 cm⁻¹ and 800 cm⁻¹ are multiplied by three for clarity. Inset: Corresponding 2D Raman images over 100 μm×100 μm area. (C-D) Working curves of adenine molecules in PBS solution with different concentrations from 1 μM to 100 μM using the adenine peak at 745 cm⁻¹ (C) before and (D) after ERS calibration.

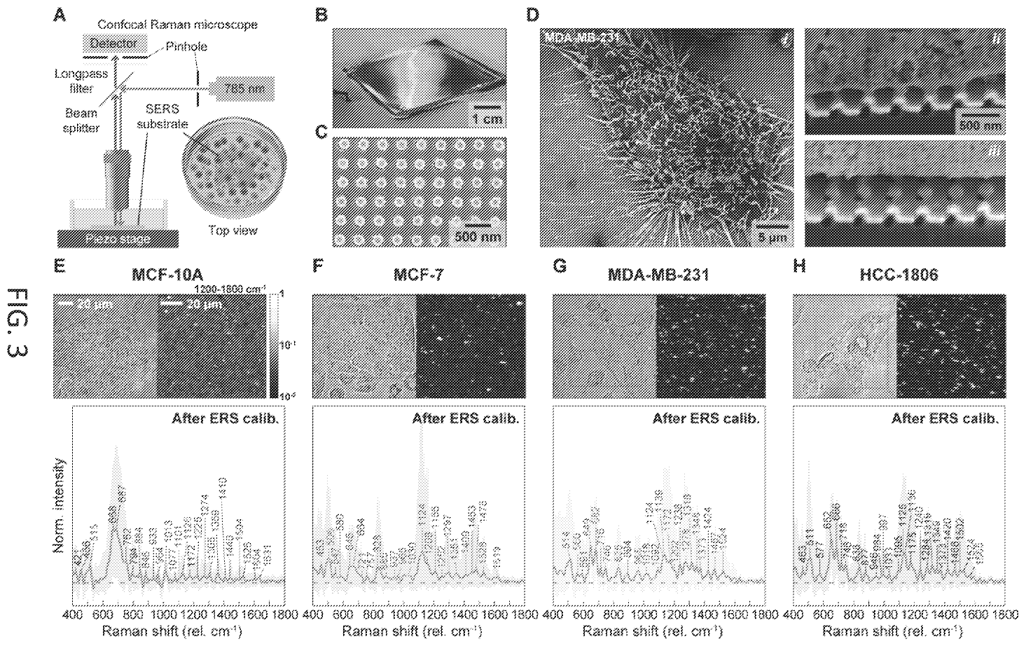

FIG. 3 shows representative data for 2D label-free SERS measurements of living breast normal and cancer cells cultured on the nanolaminated SERS substrates. (A) Schematic illustration of the experimental setup for label-free living cell SERS measurements. (B) Photograph and (C) SEM images of the nanolaminated SERS substrates. (D) (i) Top-view and (ii-iii) cross-section view of SEM images of MDA-MB-231 cultured on the nanolaminated SERS substrates. (E-H) Bright-field images (top left), 2D Raman images (top right), averaged SERS spectra of living cells after ERS calibration (bottom) for (E) MCF-10A, (F) MCF-7, (G) MDA-MB-231, and (H) HCC-1806. 2D Raman images were plotted using the integrated Raman signals of the protein-related region (1200-1800 cm⁻¹). The shaded regions in the averaged spectra are the 5th and 95th quartiles.

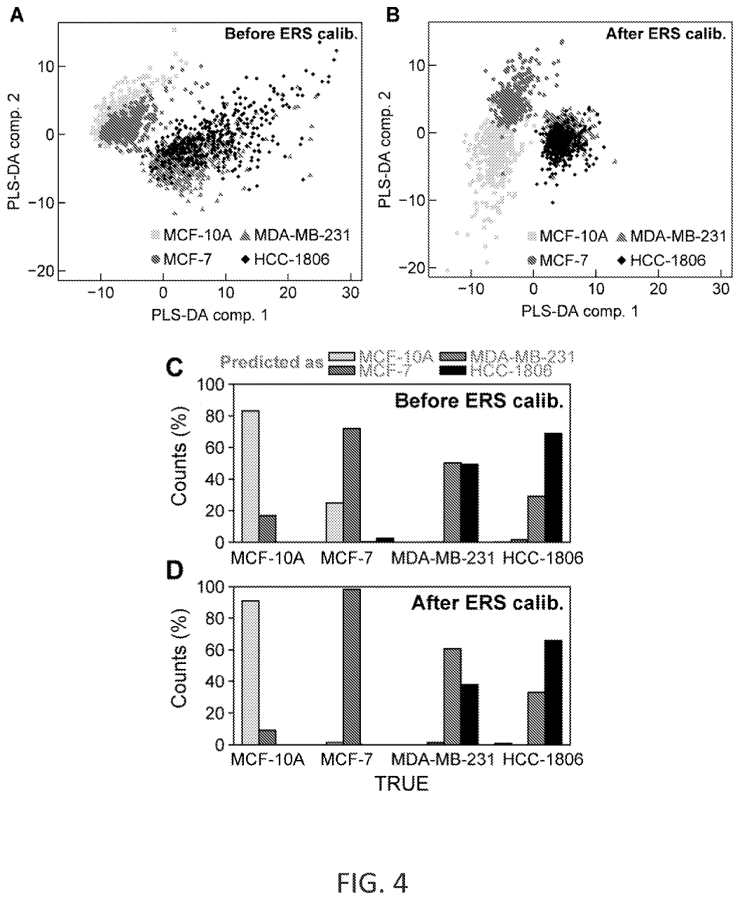

FIG. 4 shows representative data relating to improved SERS multivariate analysis by ERS calibration for subtype classification of living breast normal and cancer cells. (A-B) PLS-DA scatter plots of four different living breast normal and cancer cells (A) before and (B) after ERS calibration. (C-D) Histograms of the LOOCV confusion matrix (C) before and (D) after ERS calibration.

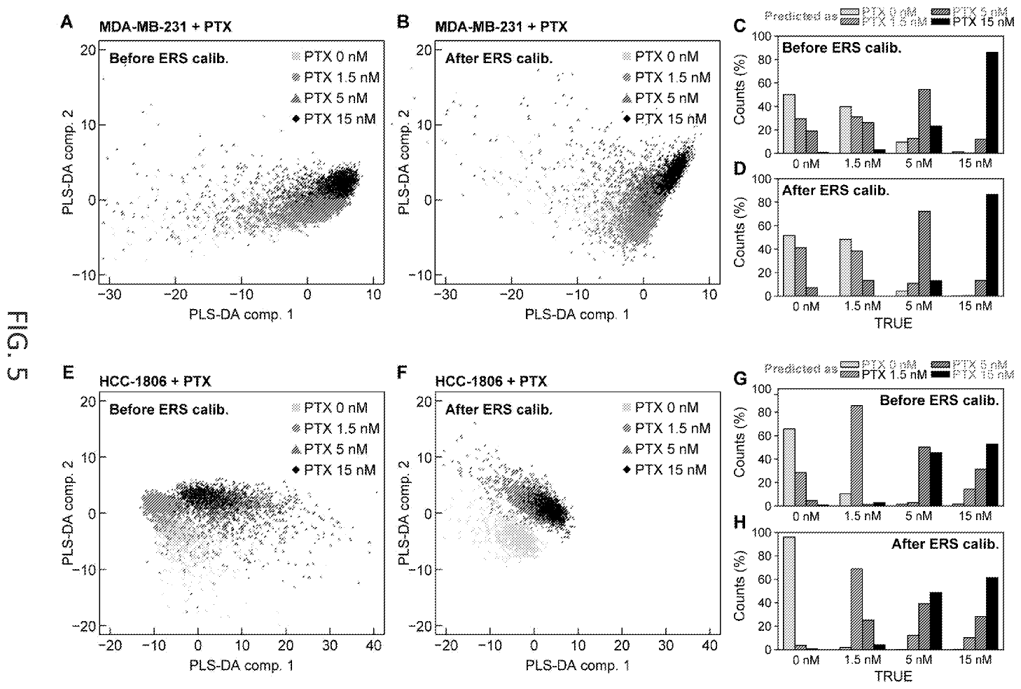

FIG. 5 shows representative data relating to average SERS multivariate analysis by ERS calibration for dosage-dependent drug efficacy study for TNBC cells. (A-B) PLS-DA scatter plots of MDA-MB-231 treated by different PTX dosages (A) before and (B) after ERS calibration. (C-D) Histograms of the LOOCV confusion matrix of the MDA-MB-231 dataset (C) before and (D) after ERS calibration. (E-F) PLS-DA scatter plots of HCC-1806 treated by different PTX dosages (E) before and (F) after ERS calibration. (G-H) Histograms of the LOOCV confusion matrix of HCC-1806 dataset (C) before and (D) after ERS calibration. IC₅₀ of PTX for each TNBC is labeled with orange color.

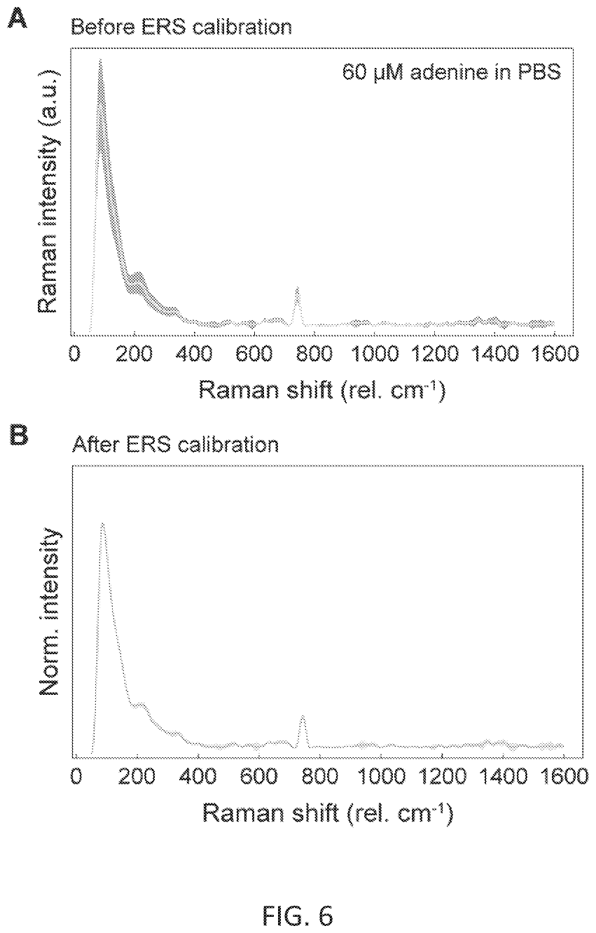

FIG. 6 shows representative data relating to average SERS spectra of 60 μM adenine solution with standard deviations (gray shaded regions) (A) before and (B) after ERS calibration. The SERS spectra are averaged from 400 pixels.

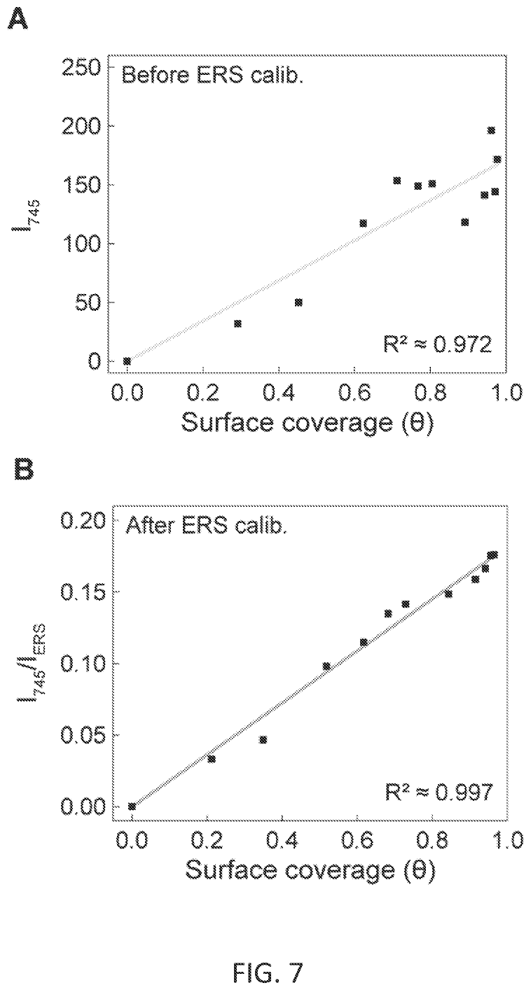

FIG. 7 shows representative data for the calculated surface coverage (8) of adenine molecules (A) before and (B) after ERS calibration.

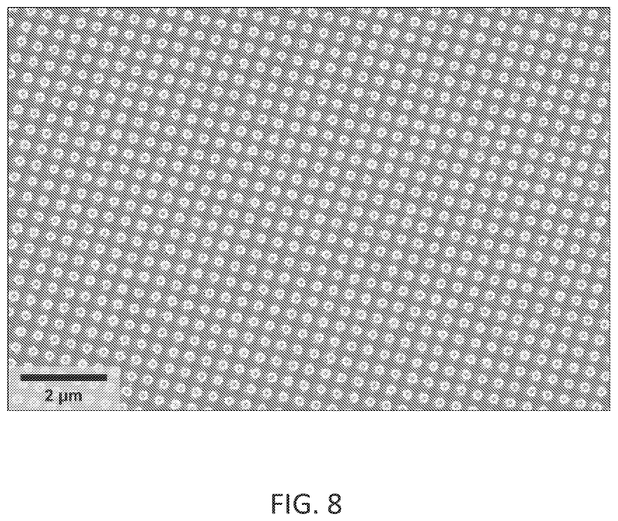

FIG. 8 shows a representative top-view SEM image of the nanolaminated SERS substrates.

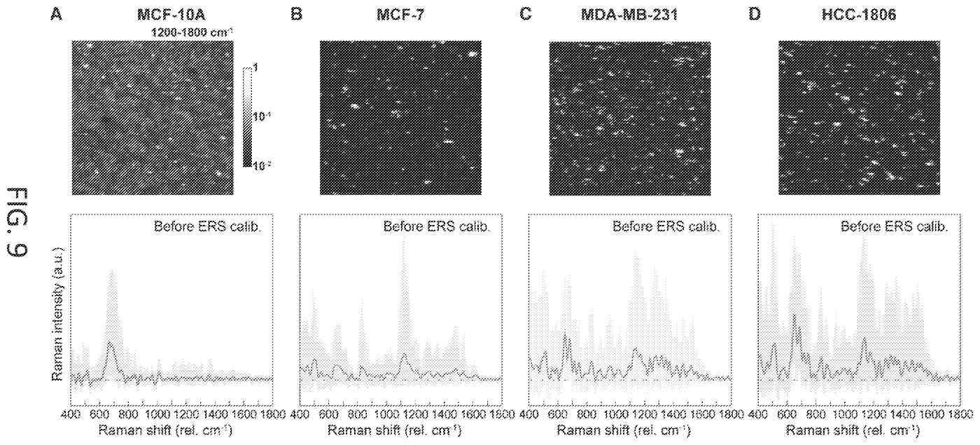

FIG. 9 shows representative data relating to 2D Raman images and SERS spectra of (A) MCF-10A, (B) MCF-7, (C) MDA-MB-231, and (D) HCC-1806 before ERS calibration.

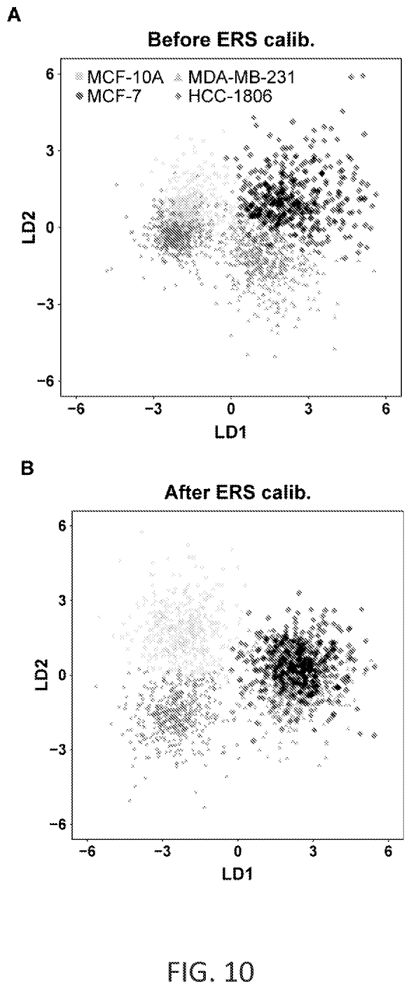

FIG. 10 shows representative data of PCA-LDA scatter plots of SERS spectra from four different living breast normal and cancer cells (A) before and (B) after ERS calibration.

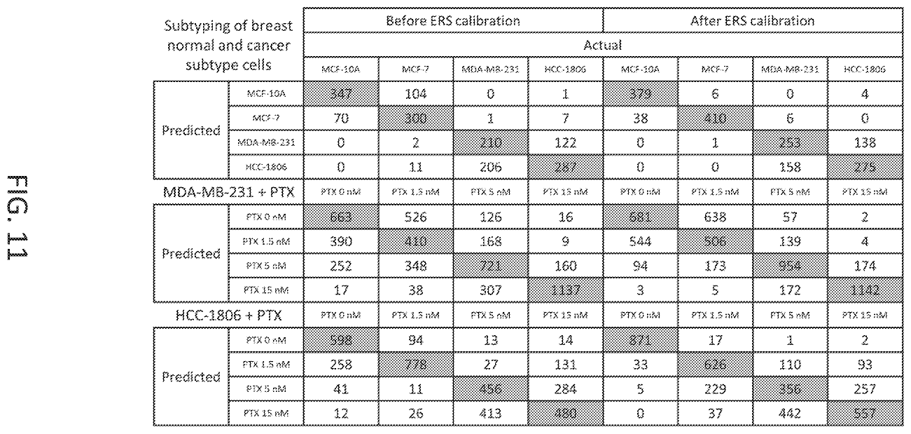

FIG. 11 shows representative data of the confusion matrix for PLS-DA models assessed by LOOCV before and after ERS calibration with raw numbers of datasets.

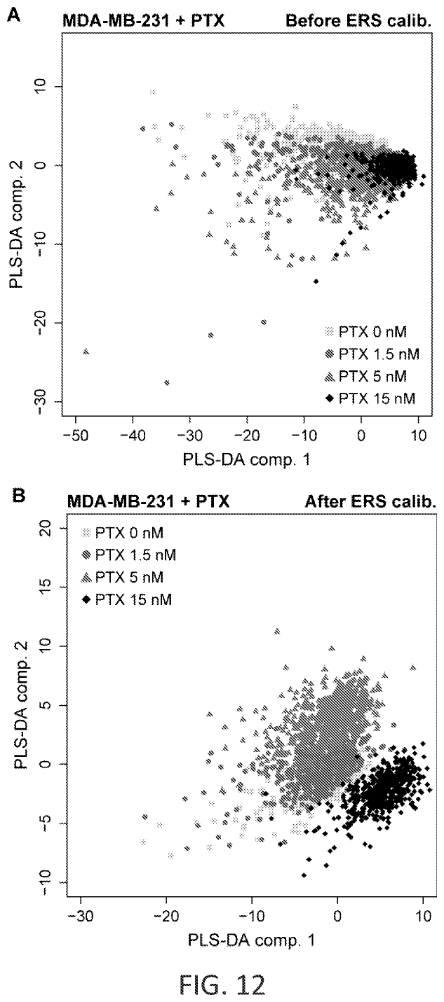

FIG. 12 shows representative data of PLS-DA scatter plots of MBA-MB-231 treated by different PTX dosages (A) before and (B) after ERS calibration.

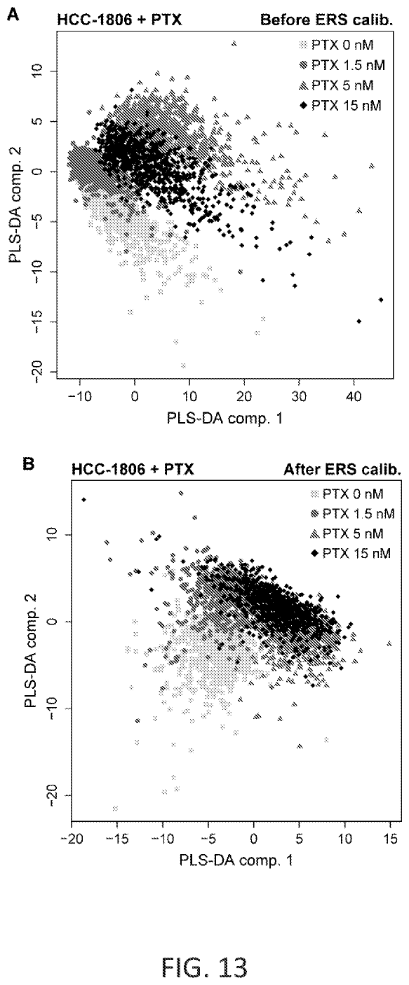

FIG. 13 shows representative data of PLS-DA scatter plots of HCC-1806 treated by different PTX dosages (A) before and (B) after ERS calibration.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosure. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### A. DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “a metal oxide,” “an inert gas,” or “a cell,” includes, but is not limited to, two or more such metal oxides, gases, or cells, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “contacting” as used herein refers to bringing a disclosed analyte, compound, chemical, or material in proximity to another disclosed analyte, compound, chemical, or material as indicated by the context. For example, a drug contacting a cell refers to the drug being in proximity to the cell by the drug interacting with the cell surface. In some instances, contacting can comprise both physical and chemical interactions between the indicated components.

As used herein, the terms “optional” or “optionally” means that the subsequently described event or circumstance can or cannot occur, and that the description includes instances where said event or circumstance occurs and instances where it does not.

As used herein, the term “analyte” refers to any substance that can be detected via SERS and which, in some embodiments, may be present in the sample. Therefore, the analyte can be, without limitation, any substance for which there exists a known chemical vibrational signal. The analyte may, for example, be an antigen, a protein, a polypeptide, a nucleic acid, a hapten, a carbohydrate, a lipid, a cell, a chemical compound, an antibody or any other of a wide variety of chemical, biological or non-biological molecules, complexes or combinations thereof.

Unless otherwise specified, temperatures referred to herein are based on atmospheric pressure (i.e. one atmosphere).

### B. ANALYTICAL CONTEXT

For acquiring intrinsic SERS signatures of living cells, two general forms of SERS-active nanosensors have been developed: colloidal plasmonic nanoparticles and substrate-based plasmonic nanostructures. Colloidal plasmonic nanoparticles, by endocytosis, can enable intracellular SERS detection and analysis of the cell death process, cell cycle, and endolysosomal pathways. On the other hand, substrate-based plasmonic nanostructures can provide uniform large-area hotspot arrays for extracellular SERS measurements to classify between cancer and normal cells, examine membrane dynamics with electroporation, and monitor neural stem cell differentiation.

Spatial variations of SERS signals among different plasmonic hotspots can occur due to variations in nanoscale geometries, local refractive index (RI) of different intracellular and extracellular components, or optical focusing conditions. Temporal variations of SERS signals can occur because of excitation laser power fluctuations or dynamic cellular perturbations to plasmonic hotspots. Such spatial or temporal variations in SERS signals can mislead interpretation of the actual biomolecule concentrations at hotspots and bias living cell SERS analysis.

Since label-free SERS spectra of living cells typically comprises highly overlapped spectroscopic features from various biomolecules in hotspot ensembles within the laser beam area, multivariate analysis of such high dimensional data is required to extract biologically meaningful knowledge. Unsupervised learning approaches, such as principal component analysis (PCA), can evaluate spectroscopic features' intrinsic relationships between sample groups and reduce data dimensionality with preserved key variances. Nevertheless, unsupervised learning algorithms are descriptive and thus necessitate further interpretation. Therefore, for interpreting high dimensional SERS spectra of living cells between different types/sub-types or disease/drug states, it is crucial to exploit supervised learning methods and perform multivariate mapping with trained models. Popular supervised learning algorithms for multivariate SERS bioanalysis include linear discriminant analysis (LDA), partial least-squares discriminant analysis (PLS-DA), support vector machine (SVM), and artificial neural network (ANN).

ERS-calibrated SERS can achieve an improved multivariate analysis of living biological systems by increasing the correlations of Raman fingerprint features with molecular concentration profiles of complex biochemical matrices at hotspots. By exploiting biocompatible Au-based plasmonic nanolaminate substrates with dense uniform hotspot arrays, SERS measurements of living cells under near-infrared (NIR) laser excitation at 785 nm can be achieved. To assess the effects of ERS-calibrated SERS on the living cell multivariate analysis performance, two supervised learning approaches can be used (e.g., PCA-LDA and PLS-DA). From the side-by-side comparison of statistical analysis results from ERS-calibrated and non-calibrated SERS datasets, the ERS calibration method improves the statistical classification accuracy in cellular subtyping. Thus, the plasmonic ERS-based calibration method can significantly boost the multivariate analysis performance in label-free SERS measurements of living biological systems and other complex biochemical matrices.

### C. NANOLAMINATED SERS SUBSTRATES

In one aspect, the invention relates to nanolaminated SERS substrates. In another aspect, the nanolaminated SERS substrate comprises vertically stacked metal-insulator-metal (MIM) nanostructures. In another aspect, the vertically stacked MIM nanostructures are on vertical nanopillar arrays.

In another aspect, the vertical nanopillar arrays are fabricated on a polyester film. In a further aspect the vertical nanopillar arrays are polymer based.

In another aspect, the vertically stacked MIM nanostructures comprise multiple layers. In a further aspect each layer independently comprises a metal or an insulator. In a further aspect, the vertically stacked MIM nanostructures comprise four metal comprising layers. In a further aspect, the vertically stacked MIM nanostructures comprise three insulator comprising layers.

In another aspect the vertically stacked MIM nanostructures comprise a metal. In another aspect, the metal is selected from Sn and Au. In another aspect, the metal is Au. In another aspect, the metal is Sn.

In a further aspect, the vertically stacked MIM nanostructures comprise an insulator. In a further aspect, the insulator is any material whose refractive index values are positive. In a further aspect, the insulator is selected from Al₂O₃, MgF₂, Indium-tin-oxide, and SiO₂. In a further aspect the insulator is SiO₂.

In a further aspect, the vertically stacked MIM nanostructures comprise a metal-insulator adhesion layer between alternating metal and insulator comprising layers. In a further aspect, the metal-insulator adhesion layer comprises Ti or Cr. In a further aspect, the metal-insulator adhesion layer comprises Ti. In a further aspect, the vertically stacked MIM nanostructures comprise a polymer-metal adhesion layer between the polymer nanopillar array and the gold. In a further aspect, the polymer-metal adhesion layer comprises Cr or Ti. In a further aspect, the polymer-metal adhesion layer comprises Cr.

In another aspect the layers of the vertically stacked MIM nanostructures comprise layers as follows, from bottom to top: nanopillar array; polymer-metal adhesion layer; layer comprising Au; metal-insulator adhesion layer; layer comprising insulator; metal-insulator adhesion layer; layer comprising Au; metal-insulator adhesion layer; layer comprising insulator; metal-insulator adhesion layer; layer comprising Au; metal-insulator adhesion layer; layer comprising insulator; metal-insulator adhesion layer; layer comprising Au.

In another aspect, the nanolaminated SERS substrate is as represented in FIG. 1D.

In another aspect the vertically stacked MIM nanostructures are etched with buffered oxide etchant.

### D. METHOD OF MANUFACTURING NANOLAMINATED SERS SUBSTRATES

One aspect of the invention is a method of manufacturing nanolaminated SERS substrates. In a further aspect, the invention is a method of manufacturing nanolaminated SERS substrates which comprise vertically stacked MIM nanostructures on vertical nanopillar arrays.

In a further aspect, the method of manufacture comprises: creating a nanopillar array; depositing seven layers onto the nanopillar array, wherein each of the layers independently comprises either a metal or an insulator, and wherein each layer is alternating from the layer below it, starting with a metal comprising layer, to form vertically stacked MIM nanostructures; and etching the vertically stacked MIM nanostructures using buffered oxide etchant.

In a further aspect, the method of manufacture comprises: creating a stamp of a nanowell array from a silicon wafer patterned with nanopillar structures; using the stamp to mold UV-curable polyurethane on a film to create a nanopillar array; curing the nanopillar array; depositing seven layers onto the nanopillar array, wherein each of the layers independently comprises either a metal or an insulator, and wherein each layer is alternating from the layer below it, starting with a metal comprising layer, to form vertically stacked MIM nanostructures; and etching the vertically stacked MIM nanostructures using buffered oxide etchant.

In a further aspect, the stamp comprises a composite polydimethylsiloxane.

In another aspect, the nanowells possess a period of about 400 nm, a diameter of about 120 nm, and a height of about 150 nm.

In another aspect, a polymer-metal adhesion layer is deposited onto the nanopillar array before the vertically stacked MIM nanostructures. In another aspect, the polymer-metal adhesion layer comprises Cr.

In another aspect, a metal-insulator adhesion layer is deposited between each of the seven layers of the vertically stacked MIM nanostructures. In another aspect, the metal-insulator adhesion layer comprises Ti.

In another aspect each metal comprising layer is between about 10 nm and 50 nm. In another aspect each metal comprising layer is between about 20 nm and 40 nm. In another aspect each metal comprising layer is between about 25 nm and 35 nm. In another aspect each metal comprising layer is about 20 nm. In another aspect each metal comprising layer is between about 40 nm. In another aspect each metal comprising layer is about 30 nm.

In a further aspect, the metal is gold.

In another aspect, each insulator comprising layer is a different thickness. In a further aspect the thickness of each insulator comprising layer increases from the bottom of the vertically stacked MIM nanostructure. In a further aspect the first applied insulator comprising layer is about 6 nm. In a further aspect the second applied insulator comprising layer is about 8 nm. In a further aspect the third applied insulator comprising layer is about 12 nm.

In a further aspect, the insulator is SiO₂.

### E. METHOD FOR PLASMONICALLY ENHANCED ELECTRONIC RAMAN SCATTERING CALIBRATED SURFACE-ENHANCED RAMAN SPECTROSCOPY

ERS processes follow the same |E_{Ioc}/Eₒ|⁴ enhancements at plasmonic hotspots, where E_{Ioc} and Eₒ are the magnitudes of local and incident electric fields, respectively, and ERS signals can act as an internal standard to calibrate SERS signals, resulting in reduced spatial and temporal variations. Plasmon-enhanced ERS intensity exponentially increases with the reduced Stokes-shifted frequency (Aw e) toward the zero value because of its linear dependence on the electron-hole pair density,

$${n}_{e - h} ( Δ {ω}_{e} ) = {\vert exp ( - \frac{ℏ Δ {ω}_{e}}{{k}_{B} T} ) - 1 \vert}^{- 1} ,$$

in metal nanostructures, where h is the Planck constant, k B is the Boltzmann constant, and T is the temperature. Therefore, in SERS measurements, by filtering Rayleigh scattering with a long pass filter, a prominent ERS pseudo-peak (FIG. 1B) can be generated as the SERS calibrator at the low-wavenumber range (<100 cm⁻¹). As derived previously, the ratio between MRS and ERS signals from the same plasmonic hotspots can be approximated as

$$\frac{{I}_{MRS}}{{I}_{ERS}} = {\vert \frac{{ε}_{M}}{{ε}_{I}} \vert}^{4} \frac{{σ}_{MRS} ( {ω}_{o} , Δ {ω}_{m} )}{{σ}_{ERS} ( {ω}_{o} , Δ {ω}_{e} )} \frac{1}{\vert {n}_{e - h} ( Δ {ω}_{e} ) + 1 \vert} \cdot r \cdot N$$

where ε_{M} and ε_{I} are the complex permittivity of metal and insulator, respectively, at the incident laser frequency ωₒ, σ_{ERS} and σ_{MRS} are the effective cross-sections for the ERS and MRS processes, respectively, Δωₘ is the Stokes-shifted frequency for the MRS process, r is the effective orientation coefficient of analyte molecules, and N is the molecular concentration. Apart from r and N, the other terms can be expressed as a material based constant M. Therefore, the ratio between MRS and ERS signals can be further approximated as I_{MRS}/I_{ERS}=M·r·N. Compared to non-calibrated SERS signals (I_{MRS}), the ERS-calibrated SERS signals (I_{MRS}/I_{ERS}) are less affected by local field variations at hotspots and can more accurately reflect the molecular concentrations in complex biochemical matrices. Notably, the plasmonically enhanced ERS signals can serve as the internal SERS calibration standard for low-uniformity SERS substrates consisting of plasmonic nanoparticle aggregations and high-uniformity nanolaminate SERS substrates.

In one aspect, the disclosed methods use plasmonically enhanced electronic Raman scattering (ERS) signals from metal nanostructures as a SERS calibration internal standard to improve multivariate analysis of living biological systems. In another aspect, the disclosed methods are capable of enhancing supervised learning classification of label-free living cell SERS spectra.

More specifically, in one aspect, the present disclosure relates to methods of label-free surface-enhanced Raman spectroscopy of cells comprising: providing a sample system, wherein the sample system comprises a nanolaminated SERS substrate, wherein a plurality of cells are adherent to at least one surface of the nanolaminated SERS substrate; carrying out ERS calibration; obtaining a dataset comprising SERS measurements over a dataset mapping area; and subjecting the dataset to multivariate analysis.

In a further aspect, the dataset mapping area is an area of between 100 μm² and 50000 μm². In a further aspect, the dataset mapping area is an area of between 1000 μm² and 45000 μm². In a further aspect, the dataset mapping area is an area of between 2000 μm² and 40000 μm². In a further aspect, the dataset mapping area is an area of between 3000 μm² and 35000 μm². In a further aspect, the dataset mapping area is an area of between 4000 μm² and 30000 μm². In a further aspect, the dataset mapping area is an area of between 5000 μm² and 20000 μm².

In a further aspect, the dataset mapping area is an area of about 100 μm². In a further aspect, the dataset mapping area is an area of about 500 μm². In a further aspect, the dataset mapping area is an area of about 1000 μm². In a further aspect, the dataset mapping area is an area of about 5000 μm², In a further aspect, the dataset mapping area is an area of about 10000 μm². In a further aspect, the dataset mapping area is an area of about 10000 μm². In a further aspect, the dataset mapping area is an area of about 20000 μm². In a further aspect, the dataset mapping area is an area of about 50000 μm².

In a further aspect, the dataset mapping area is an area of about 100 μm×100 μm. In a further aspect, the dataset mapping area contains about 20 pixels×20 pixels.

In a further aspect, the SERS measurements are obtained after near-infrared excitation over the dataset mapping area.

In a further aspect, the near-infrared excitation is carried out using a laser. In a further aspect, the near-infrared excitation is carried using a wavelength of about 700-800 nm. In a further aspect, the near-infrared excitation is carried using a wavelength of about 750 nm. In a further aspect, the near-infrared excitation is carried using a wavelength of about 785 nm.

In a further aspect, the multivariate analysis comprises a supervised machine learning method. In a further aspect, the supervised machine learning method comprises PCA-LDA. In a further aspect, the supervised machine learning method comprises PLS-DA.

In a further aspect, the vertically stacked MIM nanostructures have a RI-insensitive SERS enhancement factor greater than or equal to about 1×10⁷.

### F. CELLULAR SUBTYPING

In one aspect, the methods disclosed herein are capable of molecular-level characterization of biological samples. In one aspect, the disclosure relates to a method of multivariate analysis of cells. In another aspect, the methods provided herein are capable of providing vibrational molecular fingerprint information of biological samples without water vibrational interference. In a further aspect, the methods disclosed herein are useful for characterizing biological specimens, which may involve identifying a cell type or state corresponding to a disease or health condition of a subject.

In another aspect the vibrational molecular fingerprint information can be applied to detection of signature analytes in a complex biological system.

In another aspect the ERS-based calibration methods as described herein can be applied to any known SERS molecular profiling technique. In further aspects, the methods disclosed herein can be used to profile cell growth, cell metabolism, cell death, malignancy metrics including invasion, proliferation, and stemness, classify between cancer and normal cells, examine membrane dynamics with electroporation, or monitor neural stem cell differentiation.

In another aspect, the methods provided herein are used to identify the presence of cancer cells in a sample. In a further aspect, the methods provided herein are used to achieve cancer subtyping in a sample.

### G. MONITORING EXOGENOUS IMPACT ON CELLS

In one aspect of the invention the methods disclosed herein are capable of the statistical classification of living cells' responses to exogenous materials or stimuli.

In one aspect, the method is carried out on a first plurality of cells and a second plurality of cells, wherein the first plurality of cells has not been treated with an exogenous material or stimuli and the second plurality of cells has been treated with an exogenous material or stimuli.

In a further aspect, the exogeneous material is a drug. In a further aspect, the drug is an agent known to treat cancer. In another aspect, the agent known to treat cancer is selected from the group consisting of uracil mustard, chlormethine, cyclophosphamide, ifosfamide, melphalan, chlorambucil, pipobroman, triethylenemelamine, triethylenethiophosphoramine, busulfan, carmustine, lomustine, streptozocin, dacarbazine, temozolomide, thiotepa, altretamine, methotrexate, 5-fluorouracil, floxuridine, cytarabine, 6-mercaptopurine, 6-thioguanine, fludarabine phosphate, pentostatin, bortezomib, vinblastine, vincristine, vinorelbine, vindesine, bleomycin, dactinomycin, daunorubicin, doxorubicin, epirubicin, dexamethasone, clofarabine, cladribine, pemextresed, idarubicin, paclitaxel, docetaxel, ixabepilone, mithramycin, topotecan, irinotecan, deoxycoformycin, mitomycin-C, L-asparaginase, interferons, etoposide, teniposide 17α-ethinylestradiol, diethylstilbestrol, testosterone, prednisone, fluoxymesterone, dromostanolone propionate, testolactone, megestrolacetate, tamoxifen, methylprednisolone, methyltestosterone, prednisolone, triamcinolone, chlorotrianisene, hydroxyprogesterone, am inoglutethimide, estramustine, medroxyprogesteroneacetate, leuprolide, flutamide, toremifene, goserelin, cisplatin, carboplatin, hydroxyurea, amsacrine, procarbazine, mitotane, mitoxantrone, levamisole, navelbene, anastrazole, letrazole, capecitabine, reloxafine, droloxafine, hexamethylmelamine, oxaliplatin, gefinitib, capecitabine, erlotinib, azacitidine, temozolomide, gemcitabine, vasostatin, and combinations thereof.

In one aspect of the invention, the methods disclosed herein are capable of resolving cells' dosage-dependent responses.

In a further aspect, the method comprises at least two iterations of carrying out the method on a first plurality of cells and on a second plurality of cells; wherein in the first iteration the exogenous material is presented at a first concentration; and wherein in the second iteration the exogenous material is presented at a second concentration.

### H. REFERENCES

References are cited herein throughout using the format of reference number(s) enclosed by parentheses corresponding to one or more of the following numbered references. For example, citation of references numbers 1 and 2 immediately herein below would be indicated in the disclosure as (Refs. 1 and 2).
(1) Kneipp, J.; Kneipp, H.; Kneipp, K. Chem. Soc. Rev. 2008, 37, 1052-1060.
(2) Cialla-May, D.; Zheng, X. S.; Weber, K.; Popp, J. Chem. Soc. Rev. 2017, 46, 3945-3961.
(3) Zong, C.; Xu, M.; Xu, L. J.; Wei, T.; Ma, X.; Zheng, X. S.; Hu, R.; Ren, B. Chem. Rev. 2018, 118, 4946-4980.
(4) Heck, A. J. R. Nat. Methods 2008, 5, 927-933.
(5) Dunn, W. B.; Broadhurst, D. I.; Atherton, H. J.; Goodacre, R.; Griffin, J. L. Chem. Soc. Rev. 2011, 40, 387-426.
(6) Zheng, X. S.; Jahn, I. J.; Weber, K.; Cialla-May, D.; Popp, J. Spectrochim. Acta A Mol. Biomol. Spectrosc. 2018, 197, 56-77.
(7) Garg, A.; Nam, W.; Zhou, W. ACS Appl. Mater. Interfaces 2020, 12, 56290-56299.
(8) Song, J.; Cheng, W.; Nie, M.; He, X.; Nam, W.; Cheng, J.; Zhou, W. ACS Nano 2020, 14, 9521-9531.
(9) Nie, S. M.; Emery, S. R. Science 1997, 275, 1102-1106.
(10) Bodelon, G.; Montes-Garcia, V.; Costas, C.; Perez-Juste, I.; Perez-Juste, J.; Pastoriza-Santos, I.; Liz-Marzan, L. M. ACS Nano 2017, 11, 4631-4640.
(11) Matteini, P.; Cottat, M.; Tavanti, F.; Panfilova, E.; Scuderi, M.; Nicotra, G.; Menziani, M. C.; Khlebtsov, N.; de Angelis, M.; Pini, R. ACS Nano 2016, 11, 918-926.
(12) Morla-Folch, J.; Alvarez-Puebla, R. A.; Guerrini, L. J. Phys. Chem. Lett. 2016, 7, 3037-3041.
(13) Lussier, F.; Brule, T.; Vishwakarma, M.; Das, T.; Spatz, J. P.; Masson, J.-F. Nano Lett. 2016, 16, 3866-3871.
(14) Kneipp, J. ACS Nano 2017, 11, 1136-1141.
(15) Kuku, G.; Altunbek, M.; Culha, M. Anal. Chem. 2017, 89, 11160-11166.
(16) Laing, S.; Jamieson, L. E.; Faulds, K.; Graham, D. Nat. Rev. Chem. 2017, 1, 0060.
(17) Aioub, M.; EI-Sayed, M. A. J. Am. Chem. Soc. 2016, 138, 1258-1264.
(18) Kang, B.; Austin, L. A.; EI-Sayed, M. A. Nano Lett. 2012, 12, 5369-5375.
(19) Huefner, A.; Kuan, W. L.; Muller, K. H.; Skepper, J. N.; Barker, R. A.; Mahajan, S. ACS Nano 2016, 10, 307-316.
(20) Nam, W.; Ren, X.; Tali, S. A. S.; Ghassemi, P.; Kim, I.; Agah, M.; Zhou, W. Nano Lett. 2019, 19, 7273-7281.
(21) Ren, X.; Nam, W.; Ghassemi, P.; Strobl, J. S.; Kim, I.; Zhou, W.; Agah, M. Microsystems & Nanoengineering 2020, 6, 47.
(22) Caprettini, V.; Huang, J. A.; Moia, F.; Jacassi, A.; Gonano, C. A.; Maccaferri, N.; Capozza, R.; Dipalo, M.; De Angelis, F. Adv. Sci. 2018, 5, 1800560.
(23) EI-Said, W. A.; Kim, S. U.; Choi, J.-W. J. Mater. Chem. C 2015, 3, 3848-3859.
(24) Morais, C. L. M.; Lima, K. M. G.; Singh, M.; Martin, F. L. Nat. Protoc. 2020, 15, 2143-2162.
(25) Ringner, M. Nat. Biotech. 2008, 26, 303-304.
(26) Lussier, F.; Thibault, V.; Charron, B.; Wallace, G. Q.; Masson, J.-F. Trends in Anal. Chem. 2020, 124, 115796.
(27) Nam, W.; Zhao, Y.; Song, J.; Ali Safiabadi Tali, S.; Kang, S.; Zhu, W.; Lezec, H. J.; Agrawal, A.; Vikesland, P. J.; Zhou, W. J. Phys. Chem. Lett. 2020, 9543-9551.
(28) Kleinman, S. L.; Ringe, E.; Valley, N.; Wustholz, K. L.; Phillips, E.; Scheidt, K. A.; Schatz, G. C.; Van Duyne, R. P. J. Am. Chem. Soc. 2011, 133, 4115-4122.
(29) Kasera, S.; Biedermann, F.; Baumberg, J. J.; Scherman, O. A.; Mahajan, S. Nano Lett. 2012, 12, 5924-5928.
(30) Shen, W.; Lin, X.; Jiang, C.; Li, C.; Lin, H.; Huang, J.; Wang, S.; Liu, G.; Yan, X.; Zhong, Q.; Ren, B. Angew. Chem., Int. Ed. 2015, 54, 7308-7312.
(31) Goodacre, R.; Graham, D.; Faulds, K. Trends in Anal. Chem. 2018, 102, 359-368.
(32) Carles, R.; Bayle, M.; Benzo, P.; Benassayag, G.; Bonafos, C.; Cacciato, G.; Privitera, V. Phys. Rev. B 2015, 92, 174302.
(33) Hugall, J. T.; Baumberg, J. J. Nano Lett. 2015, 15, 2600-2604.
(34) Mertens, J.; Kleemann, M. E.; Chikkaraddy, R.; Narang, P.; Baumberg, J. J. Nano Lett. 2017, 17, 2568-2574.
(35) Song, J.; Nam, W.; Zhou, W. Adv. Mater. Technol. 2019, 4, 1800689.
(36) Liu, P. Y.; Chin, L. K.; Ser, W.; Chen, H. F.; Hsieh, C. M.; Lee, C. H.; Sung, K. B.; Ayi, T. C.; Yap, P. H.; Liedberg, B.; Wang, K.; Bourouina, T.; Leprince-Wang, Y. Lab Chip 2016, 16, 634-644.
(37) Chatterjee, A.; Cerna Sanchez, J. A.; Yamauchi, T.; Taupin, V.; Couvrette, J.; Gorodetsky, A. A. Nat. Commun. 2020, 11, 2708.
(38) Hanson, L.; Zhao, W.; Lou, H. Y.; Lin, Z. C.; Lee, S. W.; Chowdary, P.; Cui, Y.; Cui, B. Nat. Nanotechnol. 2015, 10, 554-562.
(39) Zhao, W. T.; Hanson, L.; Lou, H. Y.; Akamatsu, M.; Chowdary, P. D.; Santoro, F.; Marks, J. R.; Grassart, A.; Drubin, D. G.; Cui, Y.; Cui, B. X. Nat. Nanotechnol. 2017, 12, 750-756.
(40) Lou, H. Y.; Zhao, W. T.; Zeng, Y. P.; Cui, B. X. Acc. Chem. Res. 2018, 51, 1046-1053.
(41) Lehmann, B. D.; Bauer, J. A.; Chen, X.; Sanders, M. E.; Chakravarthy, A. B.; Shyr, Y.; Pietenpol, J. A. J. Clin. Invest. 2011, 121, 2750-2767.
(42) Iyer, S.; Gaikwad, R. M.; Subba-Rao, V.; Woodworth, C. D.; Sokolov, I. Nat. Nanotechnol. 2009, 4, 389-393.
(43) Gangoda, L.; Liem, M.; Ang, C. S.; Keerthikumar, S.; Adda, C. G.; Parker, B. S.; Mathivanan, S. Proteomics 2017, 17, 1600370.
(44) Maeda, M.; Johnson, K. R.; Wheelock, M. J. J. Cell. Sci. 2005, 118, 873-887.
(45) Altschuler, S. J.; Wu, L. F. Cell 2010, 141, 559-563.
(46) Schlegel, R. A.; Williamson, P. Cell. Death. Differ. 2001, 8, 551-563.
(47) Austin, L. A.; Kang, B.; EI-Sayed, M. A. J. Am. Chem. Soc. 2013, 135, 4688-4691.
(48) Kneipp, K.; Haka, A. S.; Kneipp, H.; Badizadegan, K.; Yoshizawa, N.; Boone, C.; Shafer-Peltier, K. E.; Motz, J. T.; Dasari, R. R.; Feld, M. S. Appl. Spectrosc. 2002, 56, 150-154.
(49) Czamara, K.; Majzner, K.; Pacia, M. Z.; Kochan, K.; Kaczor, A.; Baranska, M. J. Raman Spectrosc. 2015, 46, 4-20.
(50) Menendez, J. A.; Lupu, R. Nat. Rev. Cancer 2007, 7, 763-777.
(51) Bi, X.; Rexer, B.; Arteaga, C. L.; Guo, M.; Mahadevan-Jansen, A. J. Biomed. Opt. 2014, 19, 025001.
(52) Manciu, F. S.; Ciubuc, J. D.; Parra, K.; Manciu, M.; Bennet, K. E.; Valenzuela, P.; Sundin, E. M.; Durrer, W. G.; Reza, L.; Francia, G. Technol. Cancer. Res. Treat 2017, 16, 461-469.
(53) Movasaghi, Z.; Rehman, S.; Rehman, I. U. Appl. Spectrosc. Rev. 2007, 42, 493-541.
(54) Rygula, A.; Majzner, K.; Marzec, K. M.; Kaczor, A.; Pilarczyk, M.; Baranska, M. J. Raman Spectrosc. 2013, 44, 1061-1076.
(55) Swaminathan, V.; Mythreye, K.; O'Brien, E. T.; Berchuck, A.; Blobe, G. C.; Superfine, R. Cancer Res. 2011, 71, 5075-5080.
(56) Ballabio, D.; Consonni, V. Analytical Methods 2013, 5, 3790.
(57) Chavez, K. J.; Garimella, S. V.; Lipkowitz, S. Breast. Dis. 2010, 32, 35-48.
(58) Nieman, M. T.; Prudoff, R. S.; Johnson, K. R.; Wheelock, M. J. J. Cell Biol. 1999, 147, 631-643.
(59) Goldman, R. D.; Khuon, S.; Chou, Y. H.; Opal, P.; Steinert, P. M. J. Cell Biol. 1996, 134, 971-983.
(60) Katsumoto, T.; Mitsushima, A.; Kurimura, T. Biol. Cell 1990, 68, 139-146.
(61) Schiff, P. B.; Horwitz, S. B. Proc. Natl. Acad. Sci. 1980, 77, 1561-1565.
(62) Wang, T. H.; Wang, H. S.; Soong, Y. K. Cancer 2000, 88, 2619-2628.
(63) Weaver, B. A. Mol. Biol. Cell 2014, 25, 2677-2681.
(64) Volk-Draper, L. D.; Rajput, S.; Hall, K. L.; Wilber, A.; Ran, S. Neoplasia 2012, 14, 926-942.

The following listing of exemplary aspects supports and is supported by the disclosure provided herein.

Aspect 1. A method for label-free surface-enhanced Raman spectroscopy of cells comprising: providing a sample system, wherein the sample system comprises a nanolaminated surface-enhanced Raman spectroscopy (SERS) substrate, and wherein a plurality of cells are adherent to at least one surface of the nanolaminated SERS substrate; carrying out plasmonically enhanced electronic Raman scattering (ERS) calibration; obtaining a dataset comprising SERS measurements over a dataset mapping area; and subjecting the dataset to multivariate analysis.

Aspect 2. The method of Aspect 1, wherein the nanolaminated surface-enhanced Raman spectroscopy substrate comprises vertically stacked metal-insulator-metal (MIM) nanostructures.

Aspect 3. The method of Aspect 2, wherein the vertically stacked metal-insulator-metal (MIM) nanostructures comprise gold.

Aspect 4. The method of claim 2 or 3, wherein vertically stacked metal-insulator-metal (MIM) nanostructures having a RI-insensitive SERS enhancement factor greater than or equal to about 1×10⁷.

Aspect 5. The method of any of the foregoing claims, wherein the dataset mapping area is an area of about 100 μm×100 μm containing about 20 pixels×20 pixels.

Aspect 6. The method of any of the foregoing claims, wherein the SERS measurements are obtained after near-infrared excitation over the dataset mapping area.

Aspect 7. The method of claim 6, wherein the near-infrared excitation is carried out using a laser.

Aspect 8. The method of claim 6 or 7, wherein the near-infrared excitation is carried using a wavelength of about 700-800 nm.

Aspect 9. The method of any of the foregoing claims, wherein the multivariate analysis comprises a supervised machine learning method.

Aspect 10. The method of claim 9, wherein the supervised machine learning method comprise PCA-LDA.

Aspect 11. The method of claim 9, wherein the supervised machine learning method comprise PLS-DA.

Aspect 12. The method of any of the foregoing claims, wherein the plurality of cells comprises a cancer cell.

Aspect 13. The method of any of the foregoing claims, wherein the method is carried out on a first plurality of cells; and wherein the method is carried out on a second plurality of cells which have been treated with an exogenous material.

Aspect 14. The method of claim 13, wherein the exogeneous material is a drug.

Aspect 15. The method of claim 14, wherein the drug is an anti-cancer drug.

Aspect 16. The method of any of claims 13-15, further comprising at least two iterations of carrying out the method on a first plurality of cells and on a second plurality of cells; wherein the first iteration comprises treatment with the exogenous material at a first concentration; and wherein the second iteration comprises treatment with the exogenous material at a second concentration.

From the foregoing, it will be seen that aspects herein are well adapted to attain all the ends and objects hereinabove set forth together with other advantages which are obvious, and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and FIGS., there is no intent to limit aspects of the present disclosure to this description. On the contrary, the intent is to cover all alternatives, modifications, and equivalents included within the spirit and scope of the present disclosure.

### I. EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Cell Culture and Paclitaxel Treatment

MDA-MB-231 (American Type Culture Collection, ATCC) was grown in F12:DMEM (Dulbecco's Modified Eagle Medium, Lonza, Basel, Switzerland) with 4 mM glutamine, 10% fetal bovine serum (FBS), and penicillin-streptomycin (100 units per mL). HCC-1806 (ATCC) was grown in ATCC-formulated RPMI-1640 medium (Roswell Park Memorial Institute 1640 medium, enriched with L-glutamine, 4-(2-hydroxyethyl)-1-piperazineethanesulfonic acid (HEPES), and sodium pyruvate, ATCC 30-2001) with 10% FBS and 1% PenStrep (100 units/mL penicillium and 100 μg/mL streptomycin). MCF-7 cells (ATCC) were grown in EMEM with 10% FBS and 2×L-glutamine. MCF-10A cells (Lombardi Comprehensive Cancer Center, Georgetown University in Washington, DC) were grown in F12:DMEM with penicillin-streptomycin (100 units/mL), 20 ng/mL epidermal growth factor (EGF), 2.5 mM L-glutamine, 10 μg/mL insulin, 0.1 pg/mL cholera toxin, 0.5 μg/mL hydrocortisone, and 5% horse serum. All cells were grown in T-25 cm 2 culture flasks (Corning, NY) at 37° C. in a 5% CO2 in air atmosphere. Cells were then trypsinized and seeded on nanolaminated SERS substrates. Paclitaxel (Sigma Aldrich) was diluted in dimethyl-sulfoxide (DMSO, ATCC) with a concentration of 1.5 μM, 5 μM, and 15 μM for three different drug treatment concentrations. The solutions were mixed with 1 mL of culture medium for the final drug concentrations of 1.5 nM, 5 nM, and 15 nM. The culture medium for the control group contains the same DMSO concentration as the drug treatment medium. The control group was prepared by adding 1 μL of DMSO in 1 mL of culture medium. Once the cells were grown to 70% confluence, the medium was replaced by the new medium with paclitaxel.

### Raman Measurement

A confocal Raman microscope (Alpha 300 RSA+, WITec, Germany) for SERS measurements under laser excitation at 785 nm (Toptica Photonics, Germany) via 20×objective (NA=0.4). For 2D Raman mapping of living cells, 20×water immersion objective (NA=0.5) was used with 5 mW laser power and 20 ms integration time per pixel over 100 μm×100 μm area. Before the measurement, the instrumental calibration was verified by the silicon peak at 520 cm⁻¹. All measurements were conducted in the backscattering geometric configuration at room temperature. A long-pass filter blocks elastically scattered radiation at the wavelength corresponding to the laser line (Rayleigh scattering). Simultaneously, the rest of the collected light was guided through a multimode fiber (100 μm core diameter), acting as the pinhole for a confocal microscope, to a spectrometer (UHTS 300, WITec, Germany). The backscatter photons were dispersed with a 300 groove mm⁻¹ (750 nm blaze grating) and detected by a CCD camera (DU-401A BR-DD-352, Andor Technology, UK), which was thermoelectrically cooled and maintained at −60° C.

### Data Processing and Multivariate Analysis

Cosmic ray removal was conducted by an instrument embedded software (Project v4.1, WITec). Smoothing interpolation and data truncation were carried out with the R package hyperSpec. PCA and peak picking were done with the R packages ChemoSpec and MALDIquant, respectively. LDA and PLS-DA were performed using the R packages of MASS and mixOmics, respectively.

1. Manufacturing of Nanolaminated Sers Substrates

First, a composite polydimethylsiloxane (PDMS) stamp of nanowell arrays with a period of 400 nm, a diameter of 120 nm, and a height of 150 nm was produced from a silicon wafer patterned with nanopillar structures by soft lithography. By molding with the PDMS stamp, a UV-curable polyurethane (PU) (NOA83H, Norland Product Inc.) was used to fabricate nanopillar arrays on a flexible and optically transparent polyester film. After 10 min UV curing, an additional heat-curing process at 80° C. in a convection oven overnight was performed. Next, alternating layers of Au and SiO₂ were deposited by electron-beam deposition (PVD250, Kurt J. Lesker Company). The thickness for four Au layers is 30 nm, and the thicknesses of three SiO₂ layers are 6 nm, 8 nm, and 12 nm from bottom to top. 1 nm of Cr was deposited between the polymer nanopillar array and the first layer of Au, and 0.7 nm thick Ti between metal and insulator comprising layers as adhesion layers. Buffered oxide etchant (BOE, 10:1) (Transene Inc.) was then used to etch SiO₂ layers for 20 seconds and expose embedded MIM plasmonic hotspots.

2. FIB-SEM Characterization

FIB-SEM was performed using FEI Helios 600 Nanolab Dual-beam. Cultured cells were rinsed by PBS solution twice, followed by fixation with 2.5% glutaraldehyde in PBS solution at room temperature for 1 hour. Cells were rinsed by PBS solution twice, followed by post-fixation with 1% osmium tetroxide and dehydration in graded ethanol series from 15% to 100% (each condition was carried out for 15 min). A critical point dryer dried cells in liquid CO2. 5 nm of PtPd was sputtered as a conducting layer to reduce the charging in SEM measurements

3. Quantitative Analysis of Adenine

To demonstrate ERS calibration's effectiveness to allow quantitative SERS analysis of biomolecules, label-free SERS measurements of adenine molecules in phosphate-buffered-saline (PBS) solution with different concentrations from 1 μM to 100 μM (FIG. 2) was performed. The samples were immersed in the solutions, and 2D Raman mapping results over 100 μm×100 μm area containing 20 pixels×20 pixels were obtained. FIG. 2A shows a Raman spectrum of 60 μM adenine without ERS calibration and the corresponding 2D Raman image (inset) using a peak at 745 cm⁻¹ (ring breathing mode). A large standard deviation (SD) (gray region) and, accordingly, a large coefficient of variation (CV) value of 26% are present. On the other hand, in FIG. 2B, the ERS-calibrated SERS signals show a much smaller SD with 12% CV, and the 2D Raman image shows a more uniform intensity distribution over the large area with reduced spatial variations. Original spectra of before and after ERS calibration are available in FIG. 6. To evaluate ERS calibration improvement for quantitative analysis, the working curve from 1 μM to 100 μM using the peak at 745 cm⁻¹ (FIGS. 2C and 2D) was plotted.

The calibrated SERS signals more smoothly fit the Langmuir adsorption curve with reduced CV values for the equilibrium constant, KT, from 37.6% (4.1×105 L/mol±1.54×105 L/mol) to 11.1% (2.7×105 L/mol±0.30×105 L/mol). The SDs of all concentrations were significantly reduced with shorter error bars, and R2 values increased from 0.85 to 0.98. The surface coverage (8) of adenine molecules (FIG. 7) was calculated with the equation expressed as

$$θ = \frac{{K}_{T} \times C}{( 1 + {K}_{T} \times C )} ,$$

where C is the adenine concentration. After ERS calibration, the scatters show a better linear fitting with an improved convergence and increased R2 values from 0.972 to 0.997.

4. Subtyping of Cancer Cells

To examine the effectiveness of ERS calibration on label-free SERS analysis of living cells, 2D Raman mapping measurements of breast normal and cancer cells cultured on the nanolaminated SERS substrates were conducted and compared to the SERS profiles before and after ERS calibration (FIG. 3). Here, four different human breast normal and cancer cell lines with different degrees of malignancy were used covering a broad range of breast tumor types, including non-malignant breast normal cells (MCF-10A), moderately malignant breast cancer cells (MCF-7), and highly malignant breast cancer cells (MDA-MB-231 and HCC-1806). Both MDA-MB-231 and HCC-1806 are triple-negative breast cancer (TNBC) cells, which lack targetable receptors of progesterone (PR), estrogen (ER), and human epidermal growth factor receptor 2 (HER2). TNBC cells, therefore, have been reported to have a significantly higher risk of recurrence and the worst survival rates among subtypes of breast tumors.

FIG. 3A shows the scheme of the experimental setup. A confocal Raman microscope under backscattering configuration equipped with a 20× water immersion objective (NA=0.5) and 785 nm laser excitation was used for mapping label-free SERS spectra of living cells. To minimize time-dependent effects on the living cell SERS spectra due to the dynamic metabolic processes and stress responses to the nutrient-free environments, SERS mapping measurements were restricted within 2 hours without changing culture media with other solutions. As shown in FIGS. 3B and 3C of representative photograph and SEM images, nanolaminated SERS substrates have good nanoscale uniformity. A large-area top-view SEM image is shown in FIG. 8. FIG. 3D shows top and cross-sectional SEM images of MDA-MB-231 cultured on the nanolaminated SERS substrates. The membrane surface feature of the cultured MDA-MB-231 agrees with a previously reported study that such cancer cells reveal brush structures, consisting of microvilli and cilia with different lengths (FIG. 3D-i). Furthermore, previous reports show that vertical nanopillar structures can induce spontaneous cell engulfment, and a tight interface between the cell membrane and nanolaminated SERS substrates can improve SERS detection sensitivity. The focused ion beam (FIB) milled SEM image in FIG. 3D-ii shows that a clear nano-bio interface was formed between them, allowing direct label-free SERS measurements of cell membrane components for living cells. However, as shown in FIG. 3D-iii, some nanoantennas do not meet the cell membrane but may still detect extracellular biomolecules in their local micro-environments, such as secreted metabolites and exosomes.⁴³

FIG. 3E-H show field images, 2D images of ERS-calibrated SERS signals, and averaged Raman spectra after ERS calibration of four different living breast cells. The 2D Raman images were acquired from a 100 μm×100 μm area containing 10,000 pixels, which can accommodate a group of cells. The protein relevant range (from 1200 cm⁻¹ to 1800 cm⁻¹) was used for 2D Raman maps. Enabled by high SERS EFs (>10⁷) of the nanolaminated SERS substrates, a short integration time (20 ms) was used to collect Raman spectra with proper signal-to-noise ratios. In this way, each measurement for a Raman 2D mapping image over the large area takes only 3-5 minutes. Compared to the conventional Raman imaging, the rapid SERS spectroscopic imaging is incredibly valuable for bio-analysis of living cells by minimizing temporal deviations of molecular fingerprint information between different pixels in 2D Raman images due to dynamic cellular processes. For example, cancer cells sometimes underwent quick cell mitosis within 30-60 minutes (not shown).

By comparing 2D Raman images among different cells (the top row in FIG. 3E-H), the breast normal MCF-10A cells exhibit a more uniform signal distribution with brighter pixels than three other types of cancer cells, which reflects the inherent cellular property of MCF-10A that forms an epithelial-like compact morphology. Remarkably, despite the excellent hotspot uniformity of nanolaminated SERS substrate, there is no direct spatial correlation of cell morphologies in bright field images with 2D Raman images for different living cells, which reflects the heterogeneous, dynamic, and stochastic adsorption processes of different biomolecules at plasmonic hotspots distributed over the SERS substrates. The middle and bottom rows in FIG. 3E-H, respectively, show the average SERS spectra after ERS calibration with 5th and 95th quartiles (shaded regions) from four living breast cells. The 2D Raman images and SERS spectra before ERS calibration is shown in FIG. 9. These spectral results highlight the following critical points. First, the SERS spectra measured from all living cells show significant pixel-to-pixel variations due to the spatial heterogeneity of SERS signals from cellular biomolecules at plasmonic hotspots. Second, all living cells exhibit rich fingerprint profiles over a wide wavenumber range between 400 cm⁻¹ and 1700 cm⁻¹. Third, although there are subtle differences between spectral profiles before (FIG. 9) and after ERS calibration, it is difficult to evaluate whether ERS calibration improved the quality of living cell SERS spectra. Therefore, these observations justify using statistical approaches for analyzing the subtle differences of complex label-free SERS spectroscopic imaging data from different types of living cells.

Significantly, the average SERS spectra (FIG. 3E-H) can reveal that the measured SERS signals originate from viable living cells. First, the absence of broad carbon-based D (1350 cm⁻¹) and G (1580 cm⁻¹) bands reflects that the laser excitation conditions did not induce the photothermal graphitization of biomaterials, which can be deposited on hotspots and can mask weak SERS signals. Second, the absence of the phosphatidylserine(s) Raman signals (524 cm⁻¹, 733 cm⁻¹, and 787 cm⁻¹) from SERS hotspots in extracellular regions suggests that the measured cells are living since phosphatidylserine(s) are no longer restricted to face the inner leaflet of plasma membrane when cells undergo apoptosis. Third, the absence of Raman “death bands” of benzene ring stretching (1000 cm⁻¹) and N—H out-of-plane bending (1585 cm⁻¹) modes also reflects a healthy state of the measured cells. Finally, the DNA backbone (1125 cm⁻¹) peak appearance along with lack of adenine ring-breathing mode (735 cm⁻¹) indicate a non-denaturalized configuration of DNA from living cells.

As shown in FIG. 3E-H, all cancer cells reveal higher SERS intensities with more peaks in the lipid relevant ranges (780 cm⁻¹ to 890 cm⁻¹ and 1400 cm⁻¹ to 1550 cm⁻¹), 49 reflecting increased lipid-related components by the amplified synthesis of fatty acid and phospholipids. Similarly, all cancer cells show weak or almost no collagen peaks (815 cm⁻¹ and 852 cm⁻¹), indicating a reduced collagen feature in cancer cells. In addition, TNBC cells exhibit weak proline (855 cm⁻¹) and phospholipid (1454 cm⁻¹) intensities. A common thing for three different breast cancer cells is that they all show strong phenylalanine (621 cm⁻¹, 645 cm⁻¹, and 1170 cm⁻¹), tryptophan (879 cm⁻¹, 1208 cm⁻¹, and 1348 cm⁻¹), and tyrosine (825 cm⁻¹, 1164 cm⁻¹, and 1178 cm⁻¹) peaks compared to non-malignant cells, suggesting the increased aromatic amino acid-rich proteins on their surfaces. Remarkably, a large variations of amide III bands (1200 cm⁻¹ to 1350 cm⁻¹) from the MCF-7 cancer cells with moderate malignancy is observed as well as from the MDA-MB-231 and HCC-1806 TNBC cells with high malignancy. The observation of large amide III band variations can be associated with the disordered proteins with the beta-sheet conformation, indicating a more considerable degree of protein structural instability, i.e., less rigid and stable, consistent with the higher deformability of cancer cells.

This study used PLS-DA as a supervised classification machine learning method to maximize interclass variance among different types of cells. To investigate the effects of ERS calibration on the statistical SERS bio-analysis performance for living cells, PLS-DA was performed for the SERS dataset before and after ERS calibration (FIG. 4). Before ERS calibration, PLS-DA scatter plots in FIG. 4A shows two groups of overlapped scatters, respectively, for (1) breast normal MCF-10A cells and moderately malignant MCF-7 cancer cells and for (2) highly malignant MDA-MB-231 and HCC-1806 TNBC cells. After ERS calibration (FIG. 4B), the scatters of MCF-10A cells can be separated from those of MCF-7 cells, while the scatters of MDA-MB-231, and HCC-1806 TNBC cells still overlap due to their similar surface protein expressions. Therefore, the ERS calibration process can improve the statistical SERS bio-analysis to classify between different cell lines, suggesting that achieving a more accurate scaling of Raman fingerprint signature intensities in the measured SERS spectra from different pixels can play a positive role in the statistical analysis of biological samples.

After ERS calibration, human breast cell lines can be separated into three different groups based on the degree of malignancy: (1) non-malignant (MCF-10A); (2) moderately malignant (MCF-7); and (3) highly malignant (MDA-MB-231 and HCC-1806, TNBC cells). Subtype classification among different breast cancer cells by the degree of malignancy can be achieved due to significant molecular differences in transmembrane proteins between luminal A subtype (MCF-7) and TNBC cells, and in vimentin expression, one of the cytoskeletal components in charge of retaining cell integrity. MDA-MB-231 express vimentin, which makes it a more mesenchymal type than HCC-1806. Vimentin is typically attached to the nucleus, endoplasmic reticulum, and mitochondria. To test the generality of ERS calibration for improving statistical bio-analysis, a combination of PCA and LDA were used to process the same SERS dataset with and without ERS calibration, showing very similar results as the PLS-DA method (FIG. 10).

PLS-DA prediction abilities with and without ERS calibration can be assessed in an unbiased manner using the leave-one-out cross-validation (LOOCV) approach, FIGS. 4C and 4D visualize the confusion matrix results in histograms. FIG. 11 shows the confusion matrices of the raw numbers of spectra. Here, LOOCV was used to assume that each spectrum independently represents a specific cell type among four different cell lines. With ERS calibration, the PLS-DA prediction accuracy increases from 71% to 98% for MCF-7 and increases from 83% to 91% for MCF-10A. In contrast, after ERS calibration, MDA-MB-231 shows a prediction accuracy slightly improved from 50% to 60%, and HCC-1806 maintains a prediction accuracy around 65%, indicating that the two TNBC cell lines possess similar molecular Raman fingerprint profiles of extracellular and membrane proteins in SERS measurements.

5. Dose Response

2D SERS mapping measurements of living MDA-MB-231 and HCC-1806 cells under different PTX dosages were conducted and SERS statistical bioanalysis performance with and without ERS calibration was compared. Specifically, the cells treated with 0 nM PTX in dimethyl-sulfoxide (DMSO) serve as a control. To confirm that acquired SERS signals originate from living cells rather than chemical components of culture media or drugs, SERS measurements of bare culture media were conducted with and without PTX. As expected, due to the relatively low concentration of chemicals, no distinct Raman peaks were observed.

FIGS. 5A and 5B show PLS-DA scatter plots measured from living MDA-MB-231 cells treated with different PTX dosages before and after ERS calibration, respectively. Before ERS calibration (FIG. 5A), the scatters of the low dosage group (1.5 nM) considerably overlap with those of the control group (0 nM), while the scatters of the high dosage group (IC₅₀, 15 nM) are separated from the control group. The scatters of the middle dosage group (5 nM) distribute between the low and the high dosage groups with apparent overlaps. After ERS calibration (FIG. 5B), the separations among scatters of the low (1.5 nM), the middle (5 nM), and the high (15 nM) dosage groups are more pronounced, indicating improved molecular fingerprint profiling and bioanalysis of living cancer cell status upon drug perturbations with different dosages. On the other side, the scatters of the control (0 nM) group, and the low dosage (1.5 nM) group still have a significant overlap after ERS calibration. This observation suggests that the PTX treatment with one order of magnitude lower dosage (1.5 nM) than IC₅₀ (15 nM) is not enough to elicit significant changes of SERS-measured molecular profiles to statistically distinguish the drug effects on MDA-MB-231 cells compared to the control group (0 nM).

By comparing LOOCV confusion matrix histograms for PLS-DA results before (FIG. 5C) and after (FIG. 5D) ERS calibration, the prediction accuracy improvement in statistical SERS bioanalysis can be quantified. After ERS calibration, the prediction accuracy rate for the middle dosage (5 nM) group increases from 54% to 72%, while the prediction accuracy rate for the high dosage IC₅₀ group (15 nM) remains around 86%. For the nearly indistinguishable control (0 nM) and low dosage (1.5 nM) groups, the prediction inaccuracy rates assigned to the 5 nM and 15 nM groups are reduced significantly from 20% to 7% and from 29% to 13%, respectively. These observations imply that there may be a threshold PTX drug dosage value below 15 nM (IC₅₀) and above 5 nM and 15 nM to induce sufficient changes in SERS-measured molecular profiles to distinguish the drug effects on MDA-MB-231 cells. Future research can exploit ERS-calibrated SERS bioanalysis to investigate dynamic responses of living cells upon drug perturbations with different dosage levels.

FIGS. 5E and 5F show PLS-DA scatter plots measured from living HCC-1806 cells with different PTX dosages before and after ERS calibration, respectively. Before ERS calibration, the scatters of the low dosage group (IC₅₀, 1.5 nM) exhibit substantial overlap with the control group (0 nM), while the scatters of the middle (5 nM), and the high dosage (15 nM) groups overlap each other with separation from the control group (0 nM) and the low dosage IC₅₀ (1.5 nM) group. Remarkably, after ERS calibration, the scatters of the control group (0 nM) can completely separate from the three PTX treated groups (1.5 nM, 5 nM, and 15 nM). Among the three PTX treated groups, after ERS calibration, a gradual convergence of the scatter distributions evolving from the low dosage group (1.5 nM) to the higher dosage groups (5 nM and 15 nM) with accompanying reduced scatter distribution areas can be observed.

As shown in FIG. 5G and FIG. 5H, after ERS calibration, the control group's (0 nM) prediction accuracy rate was significantly improved from 66% to 96% with reduced overlaps of their scatters with the low dosage IC₅₀ (1.5 nM) group. In comparison, the prediction accuracy rate for the low dosage IC₅₀ (1.5 nM) group decreases from 85% to 69% due to increased overlaps of their scatters with the middle (5 nM) and the high dosage (15 nM) groups. The prediction accuracy rates for the middle (5 nM) and the high dosage (15 nM) groups do not change much after ERS calibration. In FIG. 5F, the observed converging of the scatter distributions towards the high dosage group (15 nM) is due to the drug saturation effects because the cancer cells treated with the drug dosage above IC₅₀ will have similar biological behaviors with stopped mitosis by binding PTX molecules with most microtubules. In the PLS-DA scatter plot after ERS calibration (FIG. 5F), the scatters of the low dosage IC₅₀ (1.5 nM) group have a more extensive distribution area than the higher dosage groups (5 nM and 15 nM). These observations reveal that compared to the cancer cells treated by higher dosage experiencing drug saturation effects, the population of IC₅₀ cancer cells can have a broader range of cellular biochemical states associated with the stochastic drug perturbation of cell cycle (mitotic-arrest) and apoptosis processes. Therefore, with ERS calibration, SERS molecular profiles of drug-treated living cells can allow a more accurate biostatistical analysis to distinguish dosage-dependent drug responses of living cancer cells.

6. Reproducibility

To demonstrate the reproducibility of electronic Raman scattering (ERS) calibration for surface-enhanced Raman spectroscopy (SERS) bioanalysis, label-free SERS measurements for drug responses of living breast cancer cells were performed. Triple-negative breast cancer cell lines, including MDA-MB-231 and HCC-1806, were directly cultured on nanolaminate SERS substrates. For anti-cancer drug, paclitaxel (PTX) was chosen, which is one of the most common chemotherapeutic agents for breast cancer cells, and half-maximal inhibition concentration (IC₅₀) to PTX for MDA-MB-231 and HCC-1806 are 15 nM and 1.5 nM, respectively. Four drug conditions were measured, including control (0 nM), 1.5 nM, 5 nM, and 15 nM for both cell lines, and statistical comparison of SERS bioanalysis performance with and without ERS calibration using partial least squares discriminant analysis (PLS-DA) was performed. FIGS. 12A and 12B show before and after ERS calibration scatter plots measured from living MDA-MB-231 cells treated by different PTX dosages.

Before ERS calibration (FIG. 12A), the scatters of all groups show partial overlap with each other, while two of each reveal more clustered: (1) control and the low dosage (1.5 nM) and (2) the middle (5 nM) and IC₅₀ dosage (15 nM). After ERS calibration (FIG. 12A), the separations among scatters of drug treated groups (1.5, 5, and 15 nM) are more pronounced with clearer boundaries, indicating more distinct molecular differences in response to drug dosages. On the other hand, the scatters of the control and the low dosage (1.5 nM) group exhibit more overlapping, reflecting that PTX dosage with one order of magnitude lower than IC₅₀ is insufficient to induce significant changes of extracellular profiles on MDA-MB-231 cells.

FIGS. 13A and 13B show before and after ERS calibration PLS-DA scatter plots measured from living HCC-1806 cells with different PTX dosages. Before ERS calibration, the scatters of the IC₅₀ (1.5 nM) group exhibits substantial overlap with the control group, while the scatters of the middle (5 nM) and high (15 nM) groups are distinguished. Remarkably, after ERS calibration, the degree of overlap between the control and IC₅₀ groups is decreased, and drug treated groups (1.5, 5, and 15 nM) show significant overlapping with each other. Furthermore, the control is clearly separated from all drug treated groups. Among them, we can observe a gradual convergence of the scatter distributions evolving from the low dosage (1.5 nM) to higher dosages groups (5 and 15 nM) with reduced scatter distributions. This converging trend is primarily due to the drug saturation effects since the cancer cells treated with dosages over IC₅₀ will have similar biological behaviors with limited microtubules that are interfered with PTX. The scatters of the low dosage (1.5 nM) exhibit a wider distribution than other drug treated groups (5 and 15 nM), reflecting that the cancer cells treated by IC₅₀ dosage have a broader range of cellular biochemical states associated with the stochastic drug perturbations to cell cycles.

Therefore, ERS calibration allows a more accurate biostatistical analysis to distinguish drug responses of living cancer cells. Compared to gold standard biochemical assays widely used for in vitro drug efficacy assessment, ERS-calibrated SERS bioanalysis enables non-invasive and label-free monitoring of living cells. These unique advantages to investigate dynamic cellular behaviors can potentially contribute to other living cell studies in response to external physical and chemical stimuli.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method for label-free surface-enhanced Raman spectroscopy of cells comprising:
providing a sample system;
wherein the sample system comprises a nanolaminated surface-enhanced Raman spectroscopy (SERS) substrate; and
wherein a plurality of cells are adherent to at least one surface of the nanolaminated SERS substrate;
carrying out plasmonically enhanced electronic Raman scattering (ERS) calibration;
obtaining a dataset comprising SERS measurements over a dataset mapping area;
subjecting the dataset to multivariate analysis.

2. The method of claim 1, wherein the nanolaminated surface-enhanced Raman spectroscopy substrate comprises vertically stacked metal-insulator-metal (MIM) nanostructures.

3. The method of claim 2, wherein the vertically stacked MIM nanostructures comprise gold.

4. The method of claim 3, wherein vertically stacked MIM nanostructures have a RI-insensitive SERS enhancement factor greater than or equal to about 1×10⁷.

5. The method of claim 1, wherein the dataset mapping area is an area of about 100 μm×100 μm containing about 20 pixels×20 pixels.

6. The method claim 1, wherein the SERS measurements are obtained after near-infrared excitation over the dataset mapping area.

7. The method of claim 6, wherein the near-infrared excitation is carried out using a laser.

8. The method of claim 7, wherein the near-infrared excitation is carried using a wavelength of about 700-800 nm.

9. The method of claim 1, wherein the multivariate analysis comprises a supervised machine learning method.

10. The method of claim 9, wherein the supervised machine learning method comprise PCA-LDA.

11. The method of claim 9, wherein the supervised machine learning method comprises PLS-DA.

12. The method of claim 1, wherein the plurality of cells comprises a cancer cell.

13. The method of claim 1, wherein the method is carried out on a first plurality of cells; and wherein the method is carried out on a second plurality of cells which have been treated with an exogenous material or stimuli.

14. The method of claim 13, wherein the exogeneous material is a drug.

15. The method of claim 14, wherein the drug is an anti-cancer drug.

16. The method of claim 13, further comprising at least two iterations of carrying out the method on a first plurality of cells and on a second plurality of cells; wherein the first iteration comprises treatment with the exogenous material at a first concentration; and wherein the second iteration comprises treatment with the exogenous material at a second concentration.

17. The method of claim 2, wherein vertically stacked MIM nanostructures have a RI-insensitive SERS enhancement factor greater than or equal to about 1×10⁷.

18. The method of claim 6, wherein the near-infrared excitation is carried using a wavelength of about 700-800 nm.

19. The method of claim 14, further comprising at least two iterations of carrying out the method on a first plurality of cells and on a second plurality of cells; wherein the first iteration comprises treatment with the exogenous material at a first concentration; and wherein the second iteration comprises treatment with the exogenous material at a second concentration.

20. The method of claim 15, further comprising at least two iterations of carrying out the method on a first plurality of cells and on a second plurality of cells; wherein the first iteration comprises treatment with the exogenous material at a first concentration; and wherein the second iteration comprises treatment with the exogenous material at a second concentration.
